(19) United States
(12) Patent Application Publication
(10) Pub. No.: US 2025/0129122 A1
(43) Pub. Date: Apr. 24, 2025

(54) PEPTIDE, AND CELL FUSION AGENT AND PHARMACEUTICAL COMPOSITION FOR CANCER THERAPY CONTAINING SAID PEPTIDE

(71) Applicant: Michiko KOGA, Shizuoka (JP)

(72) Inventor: Michiko KOGA, Shizuoka (JP)

(21) Appl. No.: 18/551,634

(22) PCT Filed: Mar. 22, 2022
(86) PCT No.: PCT/JP2022/013074

§ 371 Date: Nov. 13, 2023

(30) Foreign Application Priority Data
Mar. 22, 2021 (JP) 2021-047225

(51) Int. Cl. C07K 7/08; A61K 38/00; A61P 35/00; C07K 16/44

(52) U.S. Cl. CPC C07K 7/08; A61P 35/00; C07K 16/44; A61K 38/00

(57) ABSTRACT

An object of the present invention is to provide an efficient method of cell fusion, and to provide a method of killing cancer cells by cell fusion, or a method for fusing virus envelopes by membrane fusion.

The object can be solved by (1) a polypeptide comprising an amino acid sequence having a group represented by the following formula (I) on a N-terminus of an amino acid sequence selected from the group consisting of the amino acid sequences of SEQ ID NOs: 1 to 8;

—Z—Xₘ—Y (I)
wherein Z is a hydrophilic linker, X is a hydrophilic amino acid residue selected from the group consisting of serine, threonine, asparagine, glutamine, arginine, histidine, lysine, aspartic acid, glutamic acid, tyrosine, and cysteine, Y is a carboxyl group or amino group, m is an integer of 1 to 5, when m is 2 to 5, the hydrophilic amino acid residues may be the same amino acid residue or may be a combination of different amino acid residues, or (2) a polypeptide comprising an amino acid sequence having a group represented by the above formula (I) on a N-terminus of an amino acid sequence, in which 1 to 4 amino acids are deleted, substituted, inserted, and/or added in an amino acid sequence of SEQ ID NOs: 1 to 8, and having a cell fusion activity.

### CROSS REFERENCE TO RELATED APPLICATIONS

This application is a U.S. National Phase filing under 35 U.S.C. § 371 of International Application PCT/JP2022/013074, filed Mar. 22, 2022, and published as WO 2022/202785 A1 on Sep. 29, 2022. PCT/JP2022/013074 claims priority from Japanese application number 2021-047225, filed Mar. 22, 2021. The entire contents of each of these prior applications are hereby incorporated herein by reference.

### SEQUENCE LISTING

The instant application contains a Sequence Listing, which has been submitted electronically in ASCII format and is hereby incorporated by reference in its entirety. Said ASCII copy, created on Feb. 22, 2024, is named H2932878.txt and is 1,926 bytes in size.

### TECHNICAL FIELD

The present invention relates to a peptide, and cell fusion agent and pharmaceutical composition for treating cancer comprising the same. According to the present invention, cells can be efficiently fused.

### BACKGROUND ART

The cell fusion was found by the phenomenon that Sendai virus has the effect of fusing cells (Non-patent literatures 1 and 2). The cell fusion is recently used for breeding of plants, preparing monoclonal antibodies, or the like. It is known that the cell fusion is occurred by a protoplast, a PEG method, or an electrical stimulation, in addition to the use of the virus.

### CITATION LIST

### Non-Patent Literature

[NON-PATENT LITERATURE 1] Cell fusion, 1970, Harvard University Press, Mass.
[NON-PATENT LITERATURE 2] Cell Fusion and some subcellular Properties of heterokaryons and hybrids, Journal of Cell Biology, VOLUME 67, 1975, pages 257-280

### SUMMARY OF INVENTION

### Technical Problem

The present inventors have conducted studies on a method that can efficiently fuse cells, and have thought that the cell fusion could kill cancer cells.

Therefore, an object of the present invention is to provide an efficient method of cell fusion, and to provide a method of killing cancer cells by cell fusion, or a method for fusing virus envelopes by membrane fusion.

### Solution to Problem

The present inventors have conducted intensive studies into a method for efficiently fusing cells, as a result, surprisingly found that the cells can be fused by novel hydrophilic peptides having particular amino acid sequence.

Accordingly, the present invention relates to:

[1] (1) a polypeptide comprising an amino acid sequence having a group represented by the following formula (I) on a N-terminus of an amino acid sequence selected from the group consisting of the amino acid sequences of SEQ ID NOs: 1 to 8;

—Z—Xₘ—Y (I)

wherein Z is a hydrophilic linker, X is a hydrophilic amino acid residue selected from the group consisting of serine, threonine, asparagine, glutamine, arginine, histidine, lysine, aspartic acid, glutamic acid, tyrosine, and cysteine, Y is a carboxyl group or amino group, m is an integer of 1 to 5, when m is 2 to 5, the hydrophilic amino acid residues may be the same amino acid residue or may be a combination of different amino acid residues, or

(2) a polypeptide comprising an amino acid sequence having a group represented by the above formula (I) on a N-terminus of an amino acid sequence, in which 1 to 4 amino acids are deleted, substituted, inserted, and/or added in an amino acid sequence of SEQ ID NOs: 1 to 8, and having a cell fusion activity,

[2] the polypeptide of the item [1], wherein the amino acid sequence of SEQ ID NOs: 1 to 8 has methyl group at the N-terminus thereof,

[3] the polypeptide of the item [1] or [2], wherein Z is —NH—(CH₂CH₂O)ₙ—CO—, and n is an integer of 1 to 4,

[4] an antibody or an antigen binding fragment thereof, binding to the polypeptide according to any one of the items [1] to [3],

[5] a cell fusion agent comprising the polypeptide according to any one of the items [1] to [3], as an active ingredient,

[6] a pharmaceutical composition comprising the polypeptide according to any one of the items [1] to [3], as an active ingredient,

[7] the pharmaceutical composition of the item [6], for treating cancer,

[8] an anti-virus agent against a virus having envelope comprising:
(A) the polypeptide according to any one of claims 1 to 3, or
(B) (b1) a polypeptide comprising an amino acid sequence selected from the group consisting of the amino acid sequences of SEQ ID NOs: 1 to 8,
(b2) a polypeptide comprising an amino acid sequence, in which 1 to 4 amino acids are deleted, substituted, inserted, and/or added in the amino acid sequence of SEQ ID NOs: 1 to 8, and having a cell fusion activity,
(b3) a polypeptide comprising an amino acid sequence selected from the group consisting of the amino acid sequences of SEQ ID NOs: 1 to 8, and having methyl group at the N-terminus thereof, or
(b4) a polypeptide comprising an amino acid sequence, in which 1 to 4 amino acids are deleted, substituted, inserted, and/or added in the amino acid sequence of SEQ ID NOs: 1 to 8, and having a cell fusion activity, and having methyl group at the N-terminus thereof, as an active ingredient,

[9] a method for treating cancer, comprising a step of administrating to a subject in need of such treatment a therapeutically effective amount of the polypeptide according to any one of the items [1] to [3],

[10] the polypeptide according to any one of the items [1] to [3], for treating cancer, and

[11] a use of the polypeptide according to any one of the items [1] to [3], for manufacturing a pharmaceutical composition for treating cancer.

### Advantageous Effects of Invention

According to the hydrophilic polypeptide of the present invention, the cells can be efficiently fused. The hydrophilic polypeptide of the present invention can fuse the virus envelopes. In addition, the hydrophilic polypeptide of the present invention can be used as an active ingredient of the pharmaceutical composition for treating cancer.

### BRIEF DESCRIPTION OF THE DRAWINGS

FIG. 1 is micrographs (×100) of RFL cells (A) and RM4 cells (B) in the case of the treatment of RFL cells and RM4 cells by a peptide 11.

FIG. 2 is micrographs (×100) of RFL cells (A) and RM4 cells (B) in the case of the treatment of RFL cells and RM4 cells by a peptide 12.

FIG. 3 is micrographs (×100) of RFL cells (A) and RM4 cells (B) in the case of the treatment of RFL cells and RM4 cells by a peptide 13.

FIG. 4 is micrographs (×100) of RFL cells (A) and RM4 cells (B) in the case of the treatment of RFL cells and RM4 cells by a peptide 14.

FIG. 5 is micrographs (×100) of RFL cells (A) and RM4 cells (B) in the case of the treatment of RFL cells and RM4 cells by a peptide 15.

FIG. 6 is micrographs (×100) of RFL cells (A) and RM4 cells (B) in the case of the treatment of RFL cells and RM4 cells by a peptide 16.

FIG. 7 is micrographs (×100) of RFL cells (A) and RM4 cells (B) in the case of the treatment of RFL cells and RM4 cells by a peptide 17.

FIG. 8 is micrographs (×100) of RFL cells (A) and RM4 cells (B) in the case of the treatment of RFL cells and RM4 cells by a peptide 18.

FIG. 9 is an electron micrograph of RFL cells in the case of the treatment of RFL cells by a peptide 13.

FIG. 10 is a graph showing measurements of apoptosis induction by an activation of Caspase-3/7 in the case of the treatment of RFL cells by a peptide 16.

FIG. 11 is a graph showing measurements of apoptosis induction by an activation of Caspase-3/7 in the case of the treatment of RM4 cells by a peptide 16.

FIG. 12 is an electron micrograph of HVJ viruses in the case of the treatment of HVJ viruses (Sendai viruses) by a peptide 16.

FIG. 13 is 100-fold (A) and 400-fold (B) photographs of the removed tumor mass of mice treated with peptide 16, and 100-fold (C) and 400-fold (D) photographs of the removed tumor mass of control mice.

### DESCRIPTION OF EMBODIMENTS

### [1] Polypeptide

The polypeptide of the present invention comprises an amino acid sequence having a group represented by the following formula (I) on a N-terminus of an amino acid sequence selected from the group consisting of the amino acid sequences of SEQ ID NOs: 1 to 8;

—Z—Xₘ—Y (I)
wherein Z is a hydrophilic linker, X is a hydrophilic amino acid residue selected from the group consisting of serine, threonine, asparagine, glutamine, arginine, histidine, lysine, aspartic acid, glutamic acid, tyrosine, and cysteine, Y is a carboxyl group or amino group, m is an integer of 1 to 5, when m is 2 to 5, the hydrophilic amino acid residues may be the same amino acid residue or may be a combination of different amino acid residues. That is to say, in the polypeptide of the present invention, the hydrophilic amino acid residue is connected thereto through the hydrophilic linker. The binding of hydrophilic amino acids improves the solubility of the polypeptide in aqueous solution and allows efficient cell fusion.

Amino acid sequences represented by the SEQ ID NOs: 1 to 8 are as follows.

Pro-Leu-Val-Ser-Thr-Gln-Thr-Ala-Ile-Ala (SEQ ID NO:1)
Pro-Leu-Val-Ser-Thr-Gln-Thr-Ala-Leu-Ala (SEQ ID NO:2)
Pro-Leu-Val-Ser-Gln-Thr-Thr-Ala-Ile-Ala (SEQ ID NO:3)
Pro-Leu-Val-Ser-Gln-Thr-Thr-Ala-Leu-Ala (SEQ ID NO:4)
Pro-Ile-Val-Ser-Thr-Gln-Thr-Ala-Ile-Ala (SEQ ID NO:5)
Pro-Ile-Val-Ser-Thr-Gln-Thr-Ala-Leu-Ala (SEQ ID NO:6)
Pro-Ile-Val-Ser-Gln-Thr-Thr-Ala-Ile-Ala (SEQ ID NO:7)
Pro-Ile-Val-Ser-Gln-Thr-Thr-Ala-Leu-Ala (SEQ ID NO:8)

The C-terminal alanine (Ala) of the amino acid sequence represented by SEQ ID NOs: 1 to 8 is connected to the group represented by the above formula (I) by a peptide bond (—CO—NH—). In the present specification, the amino acid residue means a group of an amino acid without the OH of the carboxyl group (COOH) and without the H of the amino group (NH₂).

Z in the groups of the formula (I) is a hydrophilic linker which binds the amino acid sequence represented by SEQ ID NOs: 1 to 8 and the hydrophilic amino acid residue. By having the hydrophilic linker, the polypeptide of the amino acid sequence represented by SEQ ID NOs: 1 to 8 can exhibit the effects of the present invention, such as cell fusion activity, without being affected by the hydrophilic amino acid residue. The hydrophilic linker Z is not particularly limited, but includes hydrocarbon groups containing heteroatoms. For example, a miniPEG i.e. “—NH—(CH2CH2O)n-CO—” can be used. The above n is for example 1-4, but preferably 2-3, more preferably 2.

The hydrophilic amino acid residue is selected from serine, threonine, asparagine, glutamine, arginine, histidine, lysine, aspartic acid, glutamic acid, tyrosine, or cysteine, but preferably arginine, histidine, lysine. The number of amino acid residues, m, is 1 to 5, preferably 2 to 4. The hydrophilic amino acid residues can be L-type amino acid residues or D-type amino acid residues, but D-type amino acid residues are preferred from the viewpoint of suppressing degradation in vivo.

The amino acid residues can be the same amino acid residue or a combination of two or more amino acid residues. Specific sequence of amino acid residues includes D-Lys-D-Lys-D-Lys, D-Arg-D-Arg-D-Arg, or D-His-D-His-D-His.

In addition, the polypeptide of the present invention comprises an amino acid sequence having a group represented by the above formula (I) on a N-terminus of an amino acid sequence, in which 1 to 4 amino acids are deleted, substituted, inserted, and/or added in an amino acid sequence of SEQ ID NOs: 1 to 8, and having a cell fusion activity.

The polypeptide (1) comprising an amino acid sequence having a group represented by the above formula (I) on a N-terminus of an amino acid sequence selected from the group consisting of the amino acid sequences of SEQ ID NOs: 1 to 8, comprises a polypeptide consisting of an amino acid sequence having a group represented by the above formula (I) on a N-terminus of an amino acid sequence selected from the group consisting of the amino acid sequences of SEQ ID NOs: 1 to 8. In addition, the polypeptide (2) comprising an amino acid sequence having a group represented by the above formula (I) on a N-terminus of an amino acid sequence, in which 1 to 4 amino acids are deleted, substituted, inserted, and/or added in an amino acid sequence of SEQ ID NOs: 1 to 8, and having a cell fusion activity (hereinafter, referred to as a variation functionally equivalent) comprises a polypeptide consisting of an amino acid sequence having a group represented by the above formula (I) on a N-terminus of an amino acid sequence, in which 1 to 4 amino acids are deleted, substituted, inserted, and/or added in an amino acid sequence of SEQ ID NOs: 1 to 8, and having a cell fusion activity.

The polypeptide of the present invention can exhibit the cell fusion activity by the polypeptide consisting of sequence of ten amino acids of the amino acid sequences of SEQ ID NOs: 1 to 8 or the like, that is, the polypeptide has a specific structure for exhibiting cell fusion activity in the sequence of ten amino acids. Therefore, so long as the specific structure for exhibiting cell fusion activity in the sequence of ten amino acids is not destroyed, the polypeptide of the present invention can exhibit cell fusion activity even when other amino acid, polypeptide or protein is bonded thereto.

### <<Variation Functionally Equivalent>>

The variation functionally equivalent is not particularly limited, so long as the polypeptide comprises an amino acid sequence, in which 1 to 4 amino acids, preferably 1 to 3 amino acids, more preferably 1 to 2 amino acids, most preferably one amino acid, are deleted, substituted, inserted, and/or added in one site or plural sites of an amino acid sequence of SEQ ID NOs: 1 to 8, and has a cell fusion activity.

For example, the polypeptide comprising the amino acid sequence of SEQ ID NO: 2 or SEQ ID NO:5 is a variation functionally equivalent in which one amino acid is substituted with respect to the polypeptide comprising the amino acid sequence of SEQ ID NO: 1. The polypeptide comprising the amino acid sequence of SEQ ID NO:3 or SEQ ID NO: 6 is a variation functionally equivalent in which two amino acids are substituted with respect to the polypeptide comprising the amino acid sequence of SEQ ID NO:1. The polypeptide comprising the amino acid sequence of SEQ ID NO:4 or SEQ ID NO:7 is a variation functionally equivalent in which three amino acid are substituted with respect to the polypeptide comprising the amino acid sequence of SEQ ID NO:1. The polypeptide comprising the amino acid sequence of SEQ ID NO:8 is a variation functionally equivalent in which four amino acid are substituted with respect to the polypeptide comprising the amino acid sequence of SEQ ID NO: 1.

The “deletion, substitution, insertion, and/or addition of 1 to 4 amino acids” in the variation functionally equivalent is the “conservative substitution” that may maintain the function of the polypeptide of the present invention. The “conservative substitution” is not limited, but can be carried out by replacing an amino acid residue with different amino acid residues having similar chemical properties. As the conservative substitution, there may be mentioned, for example, a substitution of a hydrophobic residue for another hydrophobic residue, or a substitution of a polar residue for another polar residue having the same charge. Amino acids which have similar chemical properties and can be conservatively substituted with each other are known to those skilled in the art. More particularly, as nonpolar (hydrophobic) amino acids, there may be mentioned, for example, alanine, valine, isoleucine, leucine, proline, tryptophan, phenylalanine, or methionine. As polar (neutral) amino acids, there may be mentioned, for example, glycine, serine, threonine, tyrosine, glutamine, asparagine, or cysteine. As basic amino acids having a positive charge, there may be mentioned, for example, arginine, histidine, or lysine. As acidic amino acids having a negative charge, there may be mentioned, for example, aspartic acid or glutamic acid. It is not limited thereto, but the variation functionally equivalent having cell fusion activity can be obtained by such “conservative substitution.”

The polypeptide of the present invention is not limited, but preferably proline at N-terminus thereof is methylated. That is to say, the N-terminal proline is preferably methylated proline. The polypeptide of the present invention, is not limited to, but can exhibit an even excellent cell fusion activity due to the methylation of the N-terminal proline.

The polypeptide of the present invention is preferably prepared by a chemical synthesis method.

### <<Cell Fusion Activity>>

The polypeptide of the present invention has the cell fusion activity. In the cell fusion of the polypeptide of the present invention, some cells are fused and a fused cell with some nuclei are formed.

In addition, apoptosis is induced in the fused cells after cell fusion, and the cells die.

### <<Antibody>>

An antibody, such as a polyclonal antibody or a monoclonal antibody, which reacts with the polypeptide of the present invention may be obtained by directly administering the polypeptide of the present invention or a fragment thereof to various animals. Alternatively, it may be obtained by a DNA vaccine method (Raz, E. et al., Proc. Natl. Acad. Sci. USA, 91, 9519-9523, 1994; or Donnelly, J. J. et al., J. Infect. Dis., 173, 314-320, 1996), using a plasmid into which a polynucleotide encoding the polypeptide of the present invention is inserted.

The polyclonal antibody may be produced from a serum or eggs of an animal such as a rabbit, a rat, a goat, or a chicken, in which the animal is immunized and sensitized by the polypeptide of the present invention or a fragment thereof emulsified in an appropriate adjuvant (for example, Freund's complete adjuvant) by intraperitoneal, subcutaneous, or intravenous administration. The polyclonal antibody may be separated and purified from the resulting serum or eggs in accordance with conventional methods for polypeptide isolation and purification. Examples of the separation and purification methods include, for example, centrifugal separation, dialysis, salting-out with ammonium sulfate, or a chromatographic technique using such as DEAE-cellulose, hydroxyapatite, protein A agarose, and the like.

The monoclonal antibody may be easily produced by those skilled in the art, according to, for example, a cell fusion method of Kohler and Milstein (Kohler, G. and Milstein, C., Nature, 256, 495-497, 1975).

A mouse is immunized intraperitoneally, subcutaneously, or intravenously several times at an interval of a few weeks by a repeated inoculation of emulsions in which the polypeptide of the present invention or a fragment thereof is emulsified into a suitable adjuvant such as Freund's complete adjuvant. Spleen cells are removed after the final immunization, and then fused with myeloma cells to prepare hybridomas.

As a myeloma cell for obtaining a hybridoma, a myeloma cell having a marker such as a deficiency in hypoxanthine-guanine phosphoribosyltransferase or thymidine kinase (for example, mouse myeloma cell line P3X63Ag8.U1) may be used. As a fusing agent, polyethylene glycol may be used. As a medium for preparation of hybridomas, for example, a commonly used medium such as an Eagle's minimum essential medium, a Dulbecco's modified minimum essential medium, or an RPMI-1640 medium may be used by adding properly 10 to 30% of a fetal bovine serum. The fused strains may be selected by a HAT selection method. A culture supernatant of the hybridomas is screened by a well-known method such as an ELISA method or an immunohistological method, to select hybridoma clones secreting the antibody of interest. The monoclonality of the selected hybridoma is guaranteed by repeating subcloning by a limiting dilution method. Antibodies in an amount which may be purified are produced by culturing the resulting hybridomas in a medium for 2 to 4 days, or in the peritoneal cavity of a pristane-pretreated BALB/c strain mouse for 10 to 20 days.

The resulting monoclonal antibodies in the culture supernatant or the ascites may be separated and purified by conventional polypeptide isolation and purification methods. Examples of the separation and purification methods include, for example, centrifugal separation, dialysis, salting-out with ammonium sulfate, or chromatographic technique using such as DEAE-cellulose, hydroxyapatite, protein A agarose, and the like.

Further, the monoclonal antibodies or the antibody fragments containing a part thereof may be produced by inserting the whole or a part of a gene encoding the monoclonal antibody into an expression vector and introducing the resulting expression vector into appropriate host cells (such as E. coli, yeast, or animal cells).

Antibody fragments comprising an active part of the antibody such as F(ab′)2, Fab, Fab′, or Fv may be obtained by a conventional method, for example, by digesting the separated and purified antibodies (including polyclonal antibodies and monoclonal antibodies) with a protease such as pepsin, papain, and the like, and separating and purifying the resulting fragments by standard polypeptide isolation and purification methods.

Further, an antibody which reacts to the polypeptide of the present invention may be obtained in a form of single chain Fv or Fab in accordance with a method of Clackson et al. or a method of Zebedee et al. (Clackson, T. et al., Nature, 352, 624-628, 1991; or Zebedee, S. et al., Proc. Natl. Acad. Sci. USA, 89, 3175-3179, 1992). Furthermore, a humanized antibody may be obtained by immunizing a transgenic mouse in which mouse antibody genes are substituted with human antibody genes (Lonberg, N. et al., Nature, 368, 856-859, 1994).

### [2] Cell Fusion Agent

The cell fusion agent of the present invention comprises the polypeptide of the present invention as an active ingredient. The cell fusion agent of the present invention may comprise one kind of the polypeptide alone or may comprise two or more kinds of polypeptides in combination

An amount of the polypeptide in the cell fusion agent is not particularly limited, but for example, 0.1 to 100% by weight, preferably 10 to 100% by weight, more preferably 30 to 90% by weight. The cell fusion agent of the present invention may comprise, as substances other than the polypeptide, carriers (such as water or buffer), fillers, diluents, preservatives, stabilizers, antiseptics, antioxidants, or the like.

The cell fusion agent of the present invention can be used for breeding of plants, preparing monoclonal antibodies, or the like. The cell fusion agent of the present invention can effectively fuse cells.

Cells to be fused by the cell fusion agent of the present invention is not particularly limited, but includes microbial cells, plant cells, or animal cells. The animal cells include nucleated cells (for example, blood cells, lymphoid cells, visceral cells) of vertebrates (for example, mammals) such as mic, rats, rabbits, guinea pigs, goats, sheep, horses, and cows, or mammal-derived cancer cells.

A temperature of cell fusion is not particularly limited, so long as the cell fusion is induced, but for example, 0 to 40° C., preferably 10 to 38° C. A treating time is not particularly limited, but preferably 1 minute to 2 hours.

### [3] Pharmaceutical Composition

The pharmaceutical composition of the present invention comprises the polypeptide of the present invention as an active ingredient. Diseases which can be prevented or treated by the pharmaceutical composition of the present invention is not particularly limited. However, the pharmaceutical composition of the present invention can fuse the cancer cells, kill cancer cells, and treat cancers. In particular, the polypeptide of the present invention can induce apoptosis to cells by cell fusion. In the fused cells, Caspase-3/7 or AnnexinV is activated, and the apoptosis is induced. The apoptosis is induced in the fused cells, and thereby it can kill cancer cells.

The cancers that can be treated by the pharmaceutical composition of the present invention include tongue cancer, gingival cancer, malignant lymphoma, malignant melanoma, maxillary cancer, nose cancer, nasal cancer, laryngeal cancer, pharyngeal cancer, glioma, meningioma, glioma, neuroblastoma, papillary adenocarcinoma of thyroid, follicular carcinoma of thyroid, medullary carcinoma of thyroid, primary lung carcinoma, squamous cell cancer, adenocarcinoma, alveolar cell cancer, large cell undifferentiated cancer, small cell undifferentiated cancer, carcinoid, testicle tumor, prostate cancer, breast cancer, mammary Paget's disease, breast sarcoma, bone tumor, thyroid cancer, gastric cancer, liver cancer, acute myeloid leukemia, acute promedullary leukemia, acute myelomonocytic leukemia, acute monocytic leukemia, acute lymphocytic leukemia, acute undifferentiated leukemia, chronic myeloid leukemia, chronic lymphocytic leukemia, adult T-cell leukemia, malignant lymphoma, multiple myeloma, primary macroglobulinemia, childhood leukemia, esophageal cancer, gastric cancer, gastric/colon leiomyosarcoma, gastric/intestinal malignant lymphoma, pancreatic/gallbladder cancer, duodenal cancer, colon cancer, primary liver cancer, hepatoblastoma, uterine carcinoma in situ, cervical squamous epithelial carcinoma, uterine adenocarcinoma, squamous epithelial carcinoma of uterine gland, adenocarcinoma of uterine body, uterine sarcoma, uterine cancer sarcoma, destructive uterine mole, malignant chorioepithelioma of uterus, malignant melanoma of uterus, ovarian cancer, mesodermal mixed tumor, kidney cancer, renal pelvis transitional cell cancer, urinary tract transitional cell cancer, bladder papillary cancer, bladder transitional cell cancer, urinary squamous epithelial carcinoma, uretheral adenocarcinoma, Wilms's tumor, rhabdomyosarcoma, fibrosarcoma, osteosarcoma, chondrosarcoma, synovial membrane sarcoma, myxosarcoma, liposarcoma, Ewing's sarcoma, cutaneous squamous cell cancer, cutaneous basal cell cancer, Bowen's disease, Paget's disease, malignant melanoma of skin, malignant mesothelial cancer, metastatic adenocarcinoma, metastatic squamous cell cancer, metastatic sarcoma, and mesothelioma.

The formulation of the pharmaceutical composition of the present invention is not limited. However, there may be mentioned oral agents, such as powders, subtle granules, granules, tablets, capsules, suspensions, emulsions, sylups, extracts, or balls; or parenteral agents, such as injections, liquid for external use, ointments, suppositorys, creams for local administration, or eye-drops.

The above oral agent can be prepared in accordance with conventional methods, using fillers, such as gelatin, alginate sodium, starch, cornstarch, saccharose, lactose, glucose, mannitol, carboxymethyl-cellulose, dextrin, polyvinyl pyrrolidone, clystalline cellulose, soy lecithin, sucrose, fatty acid ester, talc, magnesium stearate, polyethylene glycol, magnesium silicate, silicic anhydride, or synthetic aluminum silicate; binders, disintegrators, detergents, lubricants, flow accelerator, diluents, preservatives, colorants, flavors, correctives, stabilizers, humectants, antiseptics, antioxidant, or the like.

The parenteral agents include, for example, the injections. In a preparation of the injections, an aqueous solvent such as normal saline solution or Ringer solution, non-aqueous solutions such as plant oil or fatty acid ester, a tonicity agent such as glucose or sodium chloride, a solubility assisting agent, a stabilizing agent, an antiseptic agent, a suspending agent, or an emulsifying agent, may be optionally used, in addition to the active ingredient.

A dose of the pharmaceutical composition of the present invention may be appropriately determined in accordance with, for example, age, sex, body weight, or degree of symptom of each patient, the type of each active ingredient, type of each disease, route of administration, or the like, and the determined dosage can be administered orally or parenterally. For example, in the case of an adult, the intake amount of the pharmaceutical composition of the present invention is preferably 0.01 to 100 mg/kg per day as the polypeptide. The above administration method is an example, and other administration methods may be used. It is desirable that the administration method, dose, administration period, administration interval, and the like, of the pharmaceutical composition to humans are determined by a controlled clinical trial.

In addition, dosage form for administration of the mitochondrial function activator is not limited to a drug medicine. That is, it can be administered as a food and drink of various form, such as a functional food, a healthy food (including drink), or an animal food stuff.

As a method for preparing a pharmaceutical composition containing the polypeptide, known pharmaceutical preparation methods can be used except that the polypeptide is contained as an active ingredient.

The pharmaceutical composition of the present invention may contain other components. Examples of the other components include, for example, emulsifiers such as edible fats and oils, water, glycerin fatty acid ester, sucrose fatty acid ester, sorbitan fatty acid ester, propylene glycol fatty acid ester, glycerin organic acid fatty acid ester, polyglyceryl fatty acid ester, calcium stearoyl lactylate, sodium stearoyl lactate, polyoxyethylene sorbitan fatty acid ester; thickening stabilizers such as locust bean gum, carrageenan, alginic acids, pectin, xanthan gum, crystalline cellulose, carboxymethyl cellulose, methyl cellulose, agar, glucomannan, gelatin, starch, or chemical starch; salty taste agents such as salt, or potassium chloride; acidulants such as acetic acid, lactic acid, or gluconic acid; sugars or sugar alcohols; sweeteners such as stevia or aspartame;

colorants such as beta-carotene, caramel, or red koji pigment; antioxidants such as tocopherol or tea extract; food materials or food additives such as flavoring agent; pH adjuster; food preservative, or shelf life improver. Further, the pharmaceutical composition may contain various vitamins, or functional materials such as coenzyme Q, plant sterol, or milk fat globule membrane. The amount of these other components is preferably 80% by mass or less, more preferably 40% by mass or less, and further preferably 20% by mass or less, as a total amount in the pharmaceutical composition of the present invention.

The pharmaceutical composition of the present invention can be administered to humans. Further, the subject to be administered may be animals other than human, that is, there may be mentioned pets such as dog, cat, rabbit, hamster, guinea pig, and squirrel; domestic animals such as cow, horse, and pig; experimental animals such as mouse and rat; animals bred in zoos, or the like.

### <<Method for Treating Cancer>>

The method for treating cancer of the present invention comprises a step of administrating to a subject in need of such treatment a therapeutically effective amount of the polypeptide. That is, the polypeptide of the present invention can be used for treating cancer. The cancers can be treated by administrating to humans or animals a therapeutically effective amount of the pharmaceutical composition.

### <<Polypeptide for Treating Cancer>>

The polypeptide of the present invention is for treating cancer.

The polypeptide can be used in the method for treating cancer. That is, the present specification discloses the polypeptide for treating cancer.

### <<Use of Polypeptide for Manufacturing Pharmaceutical Composition>>

The polypeptide can be used for manufacturing pharmaceutical composition. That is, the present specification discloses the use of polypeptide for manufacturing pharmaceutical composition. The pharmaceutical composition is not limited, but it is for treating cancer.

### <<Anti-Virus Agent>>

The anti-virus agent comprises (A) the polypeptide of the present invention, or (B) (b1) a polypeptide comprising an amino acid sequence selected from the group consisting of the amino acid sequences of SEQ ID NOs: 1 to 8, (b2) a polypeptide comprising an amino acid sequence, in which 1 to 4 amino acids are deleted, substituted, inserted, and/or added in the amino acid sequence of SEQ ID NOs: 1 to 8, and having a cell fusion activity, (b3) a polypeptide comprising an amino acid sequence selected from the group consisting of the amino acid sequences of SEQ ID NOs: 1 to 8, and having methyl group at the N-terminus thereof, or (b4) a polypeptide comprising an amino acid sequence, in which 1 to 4 amino acids are deleted, substituted, inserted, and/or added in the amino acid sequence of SEQ ID NOs: 1 to 8, and having a cell fusion activity, and having methyl group at the N-terminus thereof, as an active ingredient. The polypeptide can fuse viral envelopes and exhibit antiviral activity. That is, it can be used as an antiviral agent for enveloped viruses.

The enveloped viruses are not particularly limited. However, there may be mentioned viruses of Poxviridae, Baculoviridae, Rhabdoviridae, Bunyaviridae, Togaviridae, Herpesviridae, Paramyxoviridae, Orthomyxoviridae, Retroviridae, Arenaviridae, or Coronaviridae. More specifically, there may be mentioned influenza virus such as avian influenza virus, human influenza virus, swine influenza virus, hepatitis B virus, hepatitis C virus, human immunodeficiency virus, varicella-zoster virus, herpes simplex virus, human herpesvirus, mumps virus, respiratory syncytial virus, Ebola virus, rubella virus, coronavirus, measles virus, arbovirus, SARS virus, hepatitis A virus, hepatitis D Viruses, hepatitis E virus, yellow fever virus, adult T-cell leukemia virus, rabies virus, hantavirus, dengue virus, Nipah virus, or lyssavirus.

The polypeptide can be used in a method for treating the viral disease. The polypeptide can be used as a polypeptide for treating a viral disease. The polypeptide can be used for manufacturing pharmaceutical composition for virus treatment.

### <<Functions>

A mechanism wherein the polypeptide of the present invention has the cell fusion activity, has not been completely elucidated, but may be presumed to be as follows. However, the present invention is not limited by the following presumption.

The polypeptide of the present invention is considered to exhibit cell fusion activity due to the structure commonly present in the amino acid sequences of SEQ ID NOs: 1-8. It is not limited, but the first proline is considered to be relatively important. On the other hand, the polypeptides in which the 2nd leucine and 9th isoleucine are substituted with each other, show cell fusion activity, and thus the 2nd and 9th amino acids are substitutable. That is, substitutions of these amino acids with other amino acids (such as valine) are likely to exhibit cell fusion activity. Further, the polypeptides in which the 5th threonine and 6th glutamine are substituted with each other, show cell fusion activity, and thus the 5th and 6th amino acids are substitutable. That is, substitutions of these amino acids (including the 4th serine and the 7th threonine) with other amino acids having similar properties, are likely to exhibit cell fusion activity. Furthermore, it is considered that the 8th alanine and 10th alanine may also show cell fusion activity even if they are replaced with amino acids having similar properties, such as glycine. In addition, the methylation of proline at the N-terminal is not essential for the cell fusion ability of each peptide and the induction of apoptosis in cancer cells. Therefore, peptides wherein one or more amino acids are added to N-terminal proline, can also exhibit cell fusion and apoptosis-inducing ability.

A mechanism wherein the polypeptide of the present invention has the anticancer effect, has not been completely elucidated, but may be presumed to be as follows. However, the present invention is not limited by the following presumption.

It is presumed that the polypeptide of the present invention can fuse cancer cells and induce apoptosis in the cells, and thereby it can kill cancer cells. In addition, the cell fusion is induced regardless of the type of cancer. Therefore, the polypeptide of the present invention is considered to be effective against many types of cancer. Furthermore, it is considered that the polypeptide of the present invention exhibits excellent water solubility because it has the group represented by the formula (I) at the C-terminus of the amino acid sequence represented by the SEQ ID NOs: 1 to 8. The amino acid sequence represented by the SEQ ID NOs: 1 to 8 contains many hydrophobic amino acids. Therefore, it is considered that several hydrophilic amino acids are linked via a hydrophilic linker, and thereby the polypeptide as a whole exhibits excellent water solubility.

### EXAMPLES

The present invention now will be further illustrated by, but is by no means limited to, the following Examples.

### Example 1

In this synthesis example, the peptides in which the N-terminal proline of the amino acid sequences represented by the following SEQ ID NOs: 1-8 is methylated, and the peptide in which the N-terminal proline of the amino acid sequence represented by SEQ ID NO:1 is not methylated, and the peptide in which the N-terminal proline of the amino acid sequence represented by SEQ ID NO: 9 was methylated. Peptide synthesis was outsourced to Greiner/Fasmac. In connection with this, the amino acid sequence represented by SEQ ID NO:9 is an amino acid sequence in which threonine and alanine are added to the C-terminal of the amino acid sequence represented by SEQ ID NO: 1.
CH₃-Pro-Leu-Val-Ser-Thr-Gln-Thr-Ala-Ile-Ala (hereinafter referred to as a peptide 1; SEQ ID NO:1)
CH₃-Pro-Leu-Val-Ser-Thr-Gln-Thr-Ala-Leu-Ala (hereinafter referred to as a peptide 2; SEQ ID NO:2)
CH₃-Pro-Leu-Val-Ser-Gln-Thr-Thr-Ala-Ile-Ala (hereinafter referred to as a peptide 3; SEQ ID NO:3)
CH₃-Pro-Leu-Val-Ser-Gln-Thr-Thr-Ala-Leu-Ala (hereinafter referred to as a peptide 4; SEQ ID NO:4)
CH₃-Pro-Ile-Val-Ser-Thr-Gln-Thr-Ala-Ile-Ala (hereinafter referred to as a peptide 5; SEQ ID NO:5)
CH₃-Pro-Ile-Val-Ser-Thr-Gln-Thr-Ala-Leu-Ala (hereinafter referred to as a peptide 6; SEQ ID NO:6)
CH₃-Pro-Ile-Val-Ser-Gln-Thr-Thr-Ala-Ile-Ala (hereinafter referred to as a peptide 7; SEQ ID NO:7)
CH₃-Pro-Ile-Val-Ser-Gln-Thr-Thr-Ala-Leu-Ala (hereinafter referred to as a peptide 8; SEQ ID NO:8)
CH₃-Pro-Leu-Val-Ser-Thr-Gln-Thr-Ala-Ile-Ala-Thr-Ala (hereinafter referred to as a peptide 9; SEQ ID NO:9)
Pro-Leu-Val-Ser-Thr-Gln-Thr-Ala-Ile-Ala (hereinafter referred to as a peptide 10; SEQ ID NO:1)

The amino acid synthesis was carried out by the standard 9-fluorenylmethoxycarbonyl (Fmoc) method. In particular, Fmoc amino acids were activated by HBTU/HOBT solution (HBTU: 2-(1H-Benzotriazole-1-yl)-1,1,3,3-tetramethyluroniu Hexafluorophosphate; HOBT: 1-Hydroxybenzotriazole) and the amino acids were condensed by adding DIEA (N,N′-Diisopropylethylamine).

The cutout of the synthesized amino acids from the resin was carried out as follows. TFA (trifluoroacetic acid) solution (4.125 mL TFA, 0.25 mL H2O, 0.375 g phenol, 0.125 mL ethanedithiol and 0.25 mL thioanisole)) was prepared, and it was added to the resin. Then, the whole was reacted at room temperature for 2 hours and precipitated with cold ether to obtain a crude peptide.

The obtained crude peptide was purified by RP-HPLC and lyophilized. The purity of the purification was examined by HPLC and MS under the following conditions.
HPLC conditions
   A Buffer: 0.1% TFA/H2O, B Buffer: 0.1% TFA/Acetonitrile
   Column: SunFire C18 Column, 5 μm, 4.6×150 mm
   Flow rate: 1 mL/min
   Wavelength: 220 nm
MALDI-TOF-MS

### Example 2

In this synthesis example, the peptides in which the N-terminal proline of the amino acid sequences represented by the above SEQ ID NOs: 1-8 is linked with miniPEG-D-Lys-D-Lys-D-Lys-NH. Peptide synthesis was outsourced to Peptide Institute, Inc.
CH₃-Pro-Leu-Val-Ser-Thr-Gln-Thr-Ala-Ile-Ala-miniPEG-D-Lys-D-Lys-D-Lys-NH₂ (hereinafter referred to as a peptide 11)
CH₃-Pro-Leu-Val-Ser-Thr-Gln-Thr-Ala-Leu-Ala-miniPEG-D-Lys-D-Lys-D-Lys-NH₂ (hereinafter referred to as a peptide 12)
CH₃-Pro-Leu-Val-Ser-Gln-Thr-Thr-Ala-Ile-Ala-miniPEG-D-Lys-D-Lys-D-Lys-NH₂ (hereinafter referred to as a peptide 13)
CH₃-Pro-Leu-Val-Ser-Gln-Thr-Thr-Ala-Leu-Ala-miniPEG-D-Lys-D-Lys-D-Lys-NH₂ (hereinafter referred to as a peptide 14)
CH₃-Pro-Ile-Val-Ser-Thr-Gln-Thr-Ala-Ile-Ala-miniPEG-D-Lys-D-Lys-D-Lys-NH₂ (hereinafter referred to as a peptide 15)
CH₃-Pro-Ile-Val-Ser-Thr-Gln-Thr-Ala-Leu-Ala-miniPEG-D-Lys-D-Lys-D-Lys-NH₂ (hereinafter referred to as a peptide 16)
CH₃-Pro-Ile-Val-Ser-Gln-Thr-Thr-Ala-Ile-Ala-miniPEG-D-Lys-D-Lys-D-Lys-NH₂ (hereinafter referred to as a peptide 17)
CH₃-Pro-Ile-Val-Ser-Gln-Thr-Thr-Ala-Leu-Ala-miniPEG-D-Lys-D-Lys-D-Lys-NH₂ (hereinafter referred to as a peptide 18)

The “miniPEG” is-NH—(CH₂CH₂O)₂—CO—. The procedure for producing peptide 16 is described below.

Using commercially available Fmoc-Rink Amide resin (0.46 g, 0.25 mmol) as a starting material, the peptide chain was sequentially extended using the Fmoc solid phase synthesis method according to the standard method for ABI-433A to construct the desired protected peptide resin. An amino acid corresponding to miniPEG is on represented by the following formula (II):

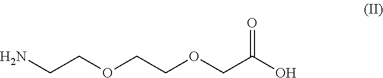 [SMILES: NCCOCCOCC(=O)O]

Then, the obtained resin was treated with trifluoroacetic acid/H2O/triisopropylsilane/1,3-dimethoxybenzene (18 mL/0.5 mL/0.5 mL/1 mL) at room temperature for 1.5 hours. The resin was filtered off, and it was decompression-concentrated. The crude peptide was solidified with ether (50 mL), purified using a reverse phase HPLC ODS column (YMC-Pack ODS-A φ30×250 mm) using water containing 0.1% trifluoroacetic acid and acetonitrile as the eluent, and freeze-dried, to obtain 180 mg of trifluoroacetate peptide. Then, the obtained trifluoroacetate peptide (180 mg) was applied to an ion exchange column (DOWEX 1×2 100-200 Mesh Anion Exchange Resin CH3COO form) using 5% aqueous acetic acid as eluent, to exchange salt. 120 mg of the target product was obtained as a white lyophilized powder of acetate salt by lyophilization.

Other peptides were also produced according to the procedure for producing peptide 16 above.

### Example 3

In this Example, the peptide 1 to 18 were added to RFL cells (rat lung fetal cells) or RM4 cells the cell fusion activity of the peptide were examined.

RFL cells or RM4 cells (2×10⁶ cells) were suspended in 6 mL of RPMI-1640 medium (Wako, 189-02025) supplemented with 5% FBS (Biosera, Cat No. 015BS493) and 8×10⁴ cells/0.25 mL were dispensed to each well of 24 well plate (Iwaki, 2820-024)), and cultured. The medium was removed, and a fresh medium (20 μL) and peptides 1 to 18 (1 μg/mL) were dispensed, and the cells were further cultured for 24 to 36 hours. After the culture was completed, the cells were fixed with methanol (Wako), and nuclear-stained with Gimza staining solution (Muto Kagaku 15003) for microscopic examination.

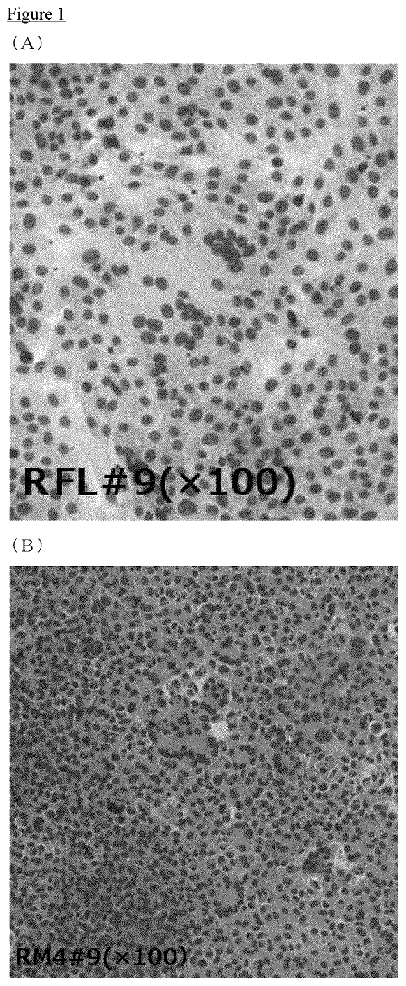

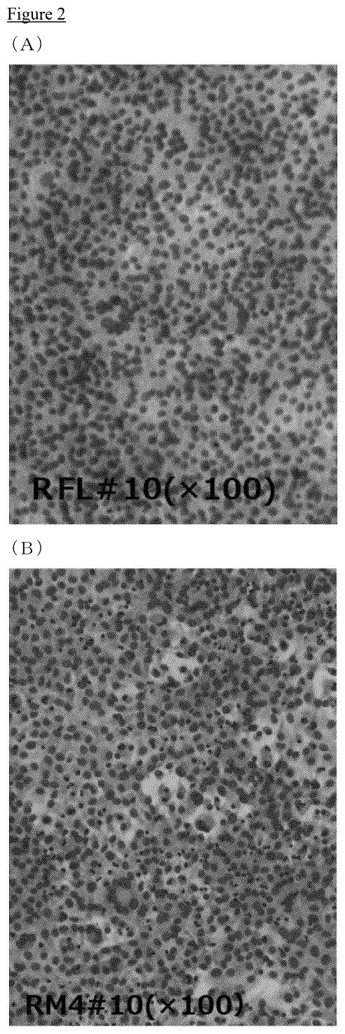

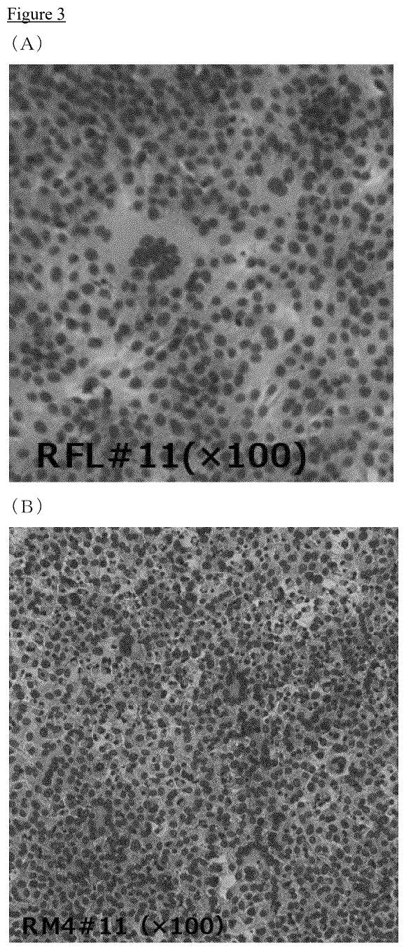

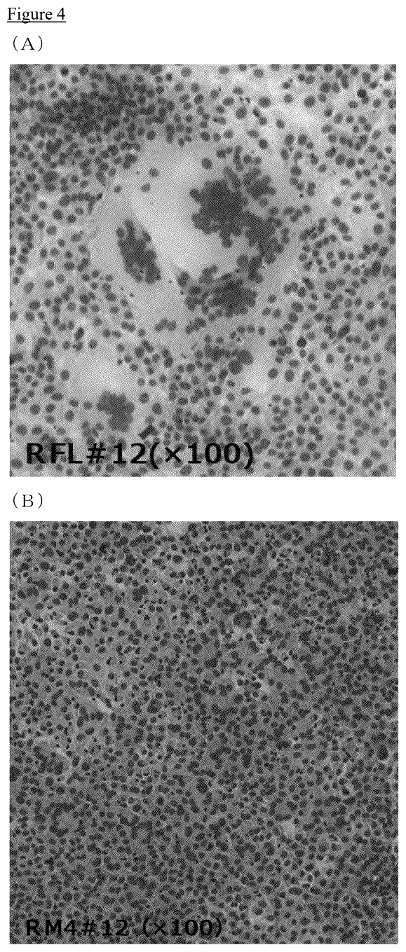

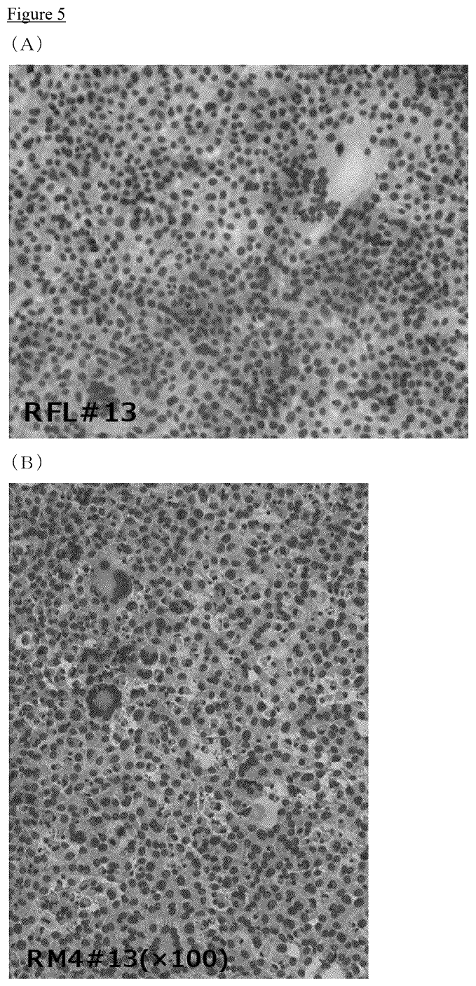

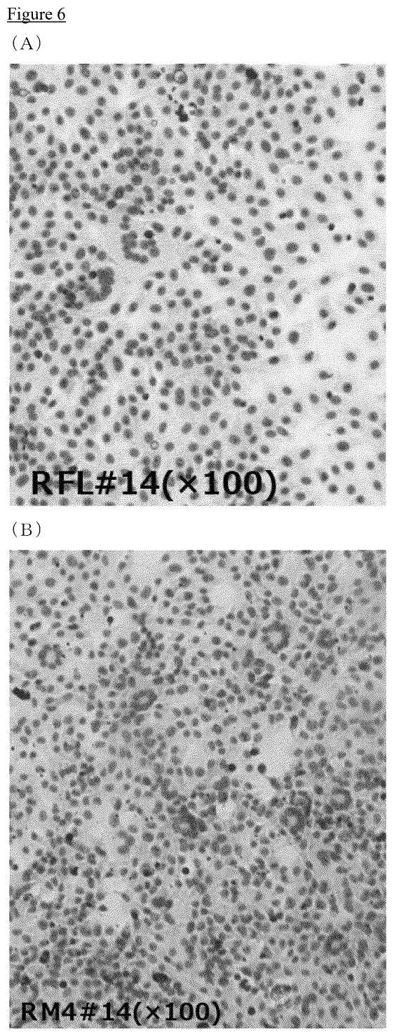

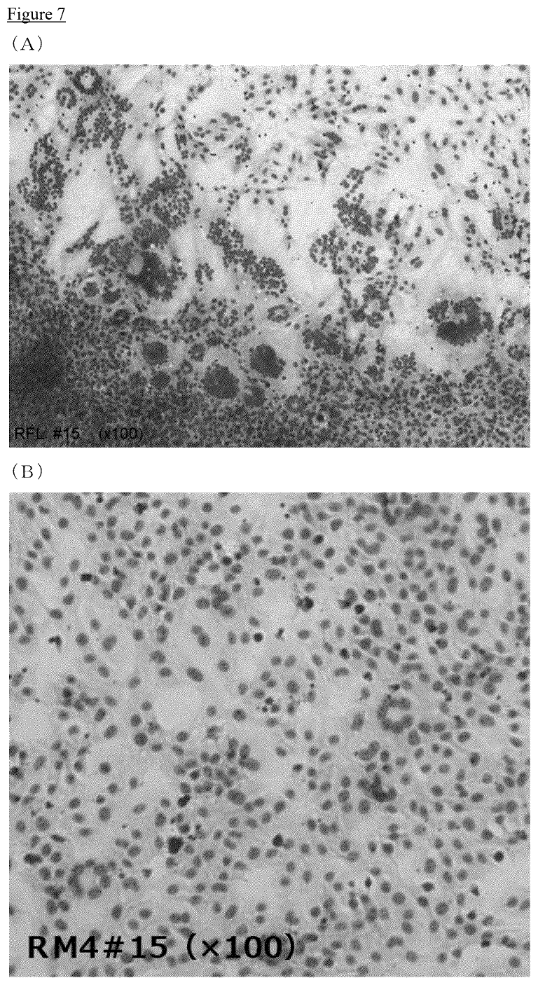

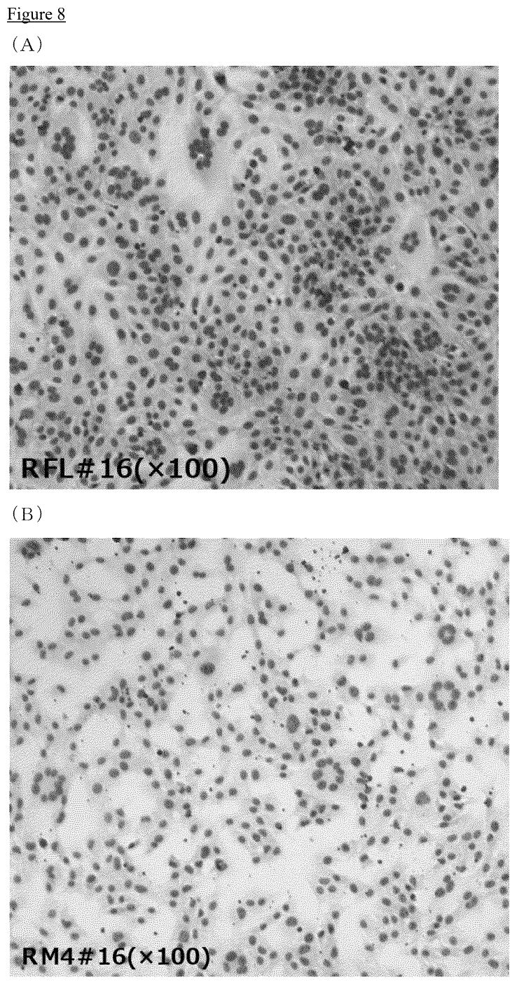

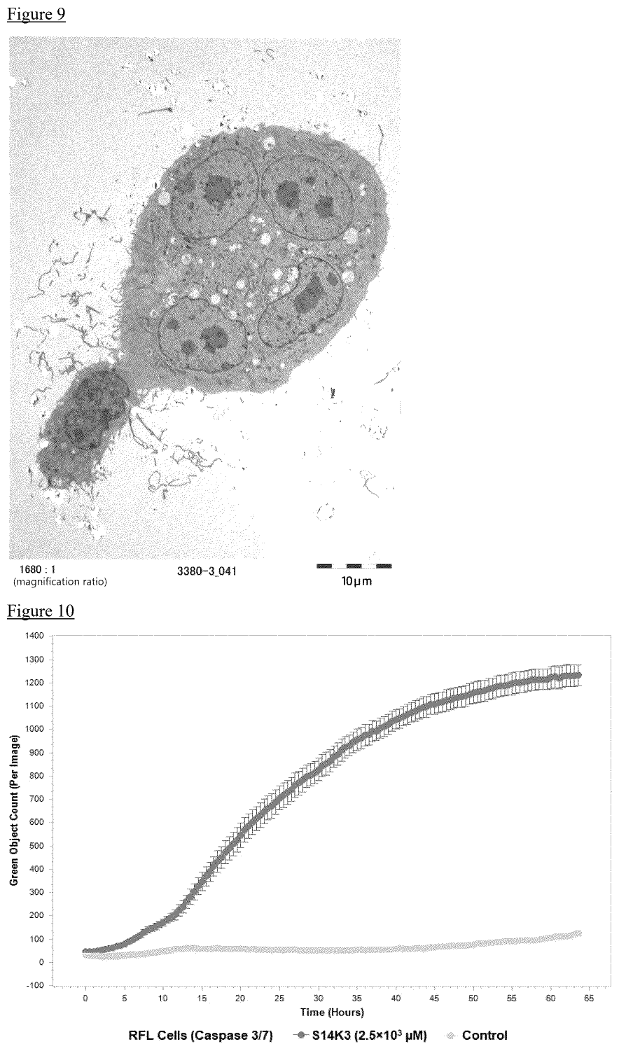

FIGS. 1 to 8 show the micrographs of RFL cells (A) and RM4 cells (B). In both RFL cells and RM4 cells, cells are fused and fusion cells with multiple nuclei were found. FIG. 9 shows an electron micrograph of RFL cells treated with peptide 16.

### Example 4

In this Example, the apoptosis ability of peptide 16 was examined using the RM-4 cells and RM4 cells. An activity of Caspase-3/7 which are indications of apoptosis, was measured using IncuCyte S3 live cell analysis system (Essen BioScience).

The activity of Caspase-3/7 activity is measured using an inert non-fluorescent (DEVD) substrate that can penetrate the cell membrane. An activated Caspase-3/7 cleaves the substrate, and thereby a DNA-bound green fluorescent label is released. The activity of Caspase-3/7 is measured by the intensity of green fluorescence.

RFL cells or RM4 cells were seeded on 96-well plates and 0.06 μg/mL of peptide 16 was added thereto. Caspase-3/7 Green Reagent (Unit size: 20 ul, 5 mM/vial) was diluted 500-fold with Ham's F-12K and added thereto.

They were measured using the IncuCyte S3 live cell analysis system, by continuously scanning every hour for 3 days with an objective lens of magnification of 10 times and 4 fields of view. As a control, RFL cells or RM-4 cells which were not treated with peptide 16, were used.

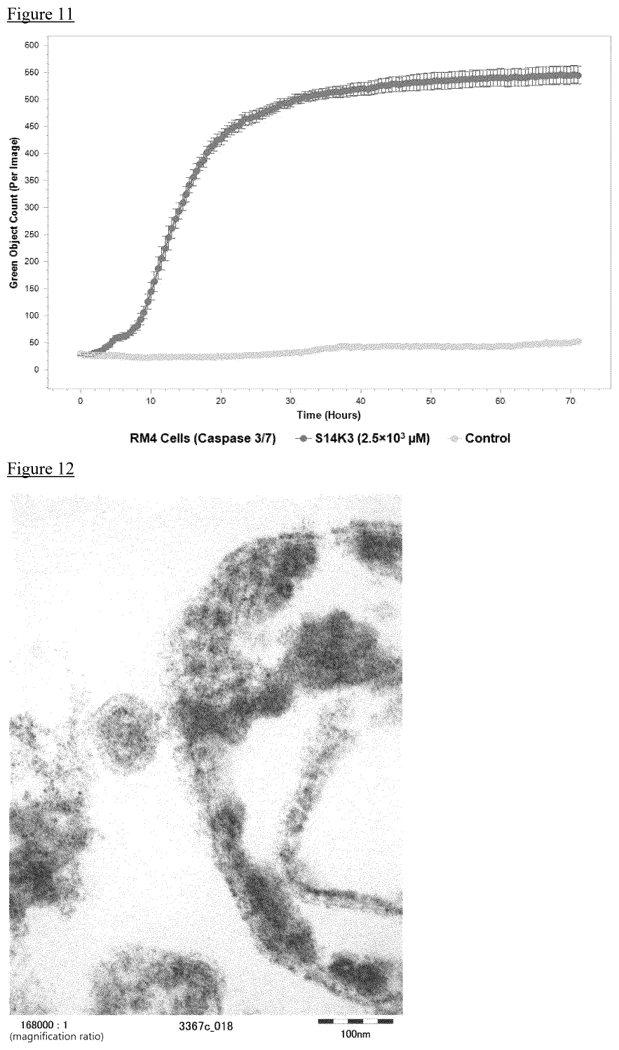

FIG. 10 shows the activity of Caspase-3/7 on RFL cells, and FIG. 11 shows the activity of Caspase-3/7 on RM4 cells. In both RFL cells and RM4 cells, the activity increased rapidly between 10 and 20 hours, and the activity gradually increased thereafter. On the other hand, in RFL cells and RM4 cells that were not treated with peptide 16, the activity of Caspase-3/7 did not increase.

### Example 5

In this example, peptide 16 was applied to the HVJ virus (Sendai virus), and its ability to fuse with the virus was examined. FIG. 12 shows an electron micrograph. Fusion of the envelope of the HVJ virus was observed.

### Example 6

In this example, the anticancer effect of peptide 16 on A549 cells (human alveolar epithelial adenocarcinoma cells) was examined in vivo.

CAnN.Cg-Foxn1nu/CrlCrlj nude mice were divided into groups of 6 animals each. A549 cells were suspended in PBS at a concentration of 1×10⁸ cells/mL and 0.1 mL thereof were transplanted subcutaneously to the right ventral part thereof. The peptide 16 was administered intravenously from the tail vein at a dose of 18.75 mg/kg or 37.5 mg/kg (peptide 16 group-1, or peptide 16 group-2) on two times/week during 5 weeks after tumor transplantation. Control group was administered PBS only.

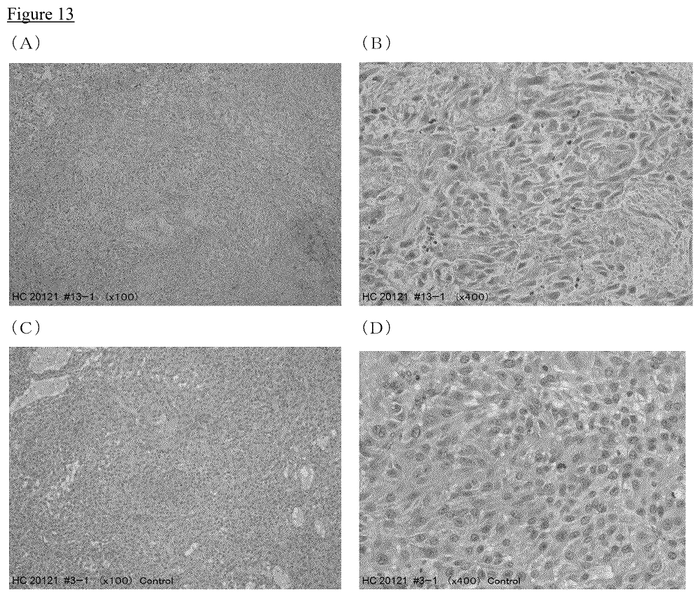

Autopsy was performed 35 days after tumor implantation, and the tumor was removed. HE-stained photographs of the excised tumors are shown in FIG. 13. FIGS. 13A (×100) and B (×400) are the photographs of tumor masses of A549 cells treated with peptide 16, and FIGS. 13C (×100) and D (×400) are the controls. The tumor tissue mass in the control is filled with cells, but the inside of the tumor is necrotic in the peptide 16-treated mice, suggesting that peptide 16 exerted an antitumor effect.

### INDUSTRIAL APPLICABILITY

The polypeptide of the present invention can be used for a cell fusion of plant cells, or animal cells. In addition, the pharmaceutical composition of the present invention can be used to the treatment of cancers.

1. (1) A polypeptide comprising an amino acid sequence having a group represented by the following formula (I) on a N-terminus of an amino acid sequence selected from the group consisting of the amino acid sequences of SEQ ID NOs: 1 to 8;
—Z—Xₘ—Y (I)
wherein Z is a hydrophilic linker, X is a hydrophilic amino acid residue selected from the group consisting of serine, threonine, asparagine, glutamine, arginine, histidine, lysine, aspartic acid, glutamic acid, tyrosine, and cysteine, Y is a carboxyl group or amino group, m is an integer of 1 to 5, when m is 2 to 5, the hydrophilic amino acid residues may be the same amino acid residue or may be a combination of different amino acid residues, or
(2) a polypeptide comprising an amino acid sequence having a group represented by the above formula (I) on a N-terminus of an amino acid sequence, in which 1 to 4 amino acids are deleted, substituted, inserted, and/or added in an amino acid sequence of SEQ ID NOs: 1 to 8, and having a cell fusion activity.

2. The polypeptide according to claim 1, wherein the amino acid sequence of SEQ ID NOs: 1 to 8 has methyl group at the N-terminus thereof.

3. The polypeptide according to claim 1, wherein Z is —NH—(CH2CH2O)n-CO—, and n is an integer of 1 to 4.

4. An antibody or an antigen binding fragment thereof, binding to the polypeptide according to claim 1.

5. A cell fusion agent comprising the polypeptide according to claim 1, as an active ingredient.

6. A pharmaceutical composition comprising the polypeptide according to claim 1, as an active ingredient.

7. The pharmaceutical composition according to claim 6, for treating cancer.

8. An anti-virus agent against a virus having envelope comprising:
(A) the polypeptide according to claim 1, or
(B) (b1) a polypeptide comprising an amino acid sequence selected from the group consisting of the amino acid sequences of SEQ ID NOs: 1 to 8,
(b2) a polypeptide comprising an amino acid sequence, in which 1 to 4 amino acids are deleted, substituted, inserted, and/or added in the amino acid sequence of SEQ ID NOs: 1 to 8, and having a cell fusion activity,
(b3) a polypeptide comprising an amino acid sequence selected from the group consisting of the amino acid sequences of SEQ ID NOs: 1 to 8, and having methyl group at the N-terminus thereof, or
(b4) a polypeptide comprising an amino acid sequence, in which 1 to 4 amino acids are deleted, substituted, inserted, and/or added in the amino acid sequence of SEQ ID NOs: 1 to 8, and having a cell fusion activity, and having methyl group at the N-terminus thereof, as an active ingredient.

9. A method for treating cancer, comprising a step of administrating to a subject in need of such treatment a therapeutically effective amount of the polypeptide according to claim 1.

10. The polypeptide according to claim 1, for treating cancer.

11. A use of the polypeptide according to claim 1, for manufacturing a pharmaceutical composition for treating cancer.


SEQUENCE LISTING (9 sequences)

SEQ ID NO: 1 - protein, 10 aa, Unknown
  Fusion peptide
PLVSTQTAIA

SEQ ID NO: 2 - protein, 10 aa, Unknown
  Fusion peptide
PLVSTQTALA

SEQ ID NO: 3 - protein, 10 aa, Unknown
  Fusion peptide
PLVSQTTAIA

SEQ ID NO: 4 - protein, 10 aa, Unknown
  Fusion peptide
PLVSQTTALA

SEQ ID NO: 5 - protein, 10 aa, Unknown
  Fusion peptide
PIVSTQTAIA

SEQ ID NO: 6 - protein, 10 aa, Unknown
  Fusion peptide
PIVSTQTALA

SEQ ID NO: 7 - protein, 10 aa, Unknown
  Fusion peptide
PIVSQTTAIA

SEQ ID NO: 8 - protein, 10 aa, Unknown
  Fusion peptide
PIVSQTTALA

SEQ ID NO: 9 - protein, 12 aa, Unknown
  Fusion peptide
PLVSTQTAIATA